(19) United States
(12) Patent Application Publication
(10) Pub. No.: US 2010/0010228 A1
(43) Pub. Date: Jan. 14, 2010

(54) PROCESS FOR THE ASYMMETRIC REDUCTION OF 3-BENZOYLPIPERIDINES

(75) Inventor: Mark B. Mitchell, Durham, NC (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Vitae Pharmaceuticals, Inc., Fort Washington, PA (US)

(21) Appl. No.: 12/490,947

(22) Filed: Jun. 24, 2009

Related U.S. Application Data
(60) Provisional application No. 61/075,832, filed on Jun. 26, 2008.

(51) Int. Cl. C07D 211/22; C07D 211/32

(52) U.S. Cl. 546/245

(57) ABSTRACT

Disclosed is a process for the asymmetric preparation of a phenyl(3-piperidinyl)methanol represented by Formula (I):

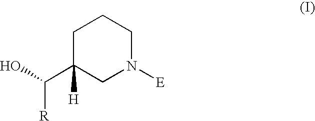

wherein R is an optionally substituted phenyl and E is an amine protecting group.

### RELATED APPLICATION

This application claims the benefit of U.S. Provisional Application No. 61/075,832, filed on Jun. 26, 2008.

The entire teachings of the above application are incorporated herein by reference.

### BACKGROUND OF THE INVENTION

Compounds of Formula (I) are useful intermediates in the preparation of compounds having inhibitory activity against aspartic proteases, particularly renin, as described in International Publications WO 2006/042150, WO 2007/070201, WO 2007/117557, WO 2007/117560, WO 2008/036216, and WO 2008/036247. Significant quantities of the pure aspartic protease/renin inhibitor are required in the drug development process, e.g., for in vitro and in vivo testing. Accordingly, it would be useful to develop efficient processes for the large-scale preparation of intermediates employed in the synthesis of such aspartic protease/renin inhibitor compounds.

Naud catalyst, (S)-2-[(Sₚ)-2-(diphenylphosphino)ferrocenyl]-4-isopropyl-2-oxazoline triphenylphosphine ruthenium(II) chloride complex has been shown to effect the asymmetric hydrogenation of aryl ketones in good yield and high enantioselectivity. However, the efficacy ofthis hydrogenation catalyst has been demonstrated primarily for simple aryl methyl ketones. There are only a few examples of hydrogenations of ketones possessing α-substitution on the methyl group employing the Naud catalyst. There are no examples of the use of this catalyst (or its enantiomer) for selectively hydrogenating ketone substrates that are disubstituted at the ketone α-position where that disubstitution comprises a saturated heterocyclic moiety.

It has also been reported that the addition of a strong base is essential to the catalytic activity of the Naud catalyst. This may be problematic in the reduction of certain a-disubstituted arylmethyl ketones as the chiral center at the α-position of the ketone substrate may potentially racemize under basic conditions.

### SUMMARY OF THE INVENTION

This invention is directed to a process for the asymmetric preparation of a phenyl(3-piperidinyl)methanol represented by Formula (I):

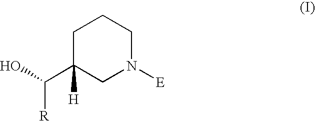

wherein R is phenyl optionally substituted with 1 to 3 groups independently selected from:
1) halogen, (C₁-C₆)alkyl, (C₃-C₆)cycloalkyl, halo(C₁-C₆)alkyl, (C₁-C₆)alkoxy, and (C₃-C₆)cycloalkoxy; and
2) phenyl, heteroaryl, phenoxy, and heteroaryloxy, each optionally substituted with 1 to 2 groups independently selected from: halogen, (C₁-C₄)alkyl, halo(C₁-C₄)alkyl, (C₁-C₄)alkoxy, and (C₁-C₄)alkoxy(C₁-C₄)alkyl; and
E is an amine protecting group;
   wherein the process comprises hydrogenating a ketone of Formula (II):

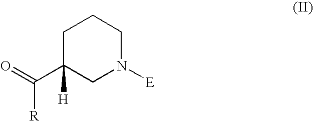

under hydrogen gas pressure in the presence of a base and a ruthenium catalyst.

### DETAILED DESCRIPTION OF THE INVENTION

In the processes of this invention, when E is an amine protecting group, it is understood the E may be any amine protecting group that is compatible with the processes of this invention. Such amine protecting groups are well-known in the art (See T. W. Greene and P. G. M. Wuts “Protective Groups in Organic Synthesis” John Wiley & Sons, Inc., New York 1999). For example, E may be selected from a carbamate, amide, formate, sulfonamide, alkyl, or benzyl protecting group. Exemplary amine protecting groups include tert-butoxycarbonyl (Boc), benzyloxycarbonyl (Cbz), and 1-[2-(trimethylsilyl)ethoxycarbonyl] (Teoc).

“Alkyl” means a saturated aliphatic branched or straight-chain mono- or di-valent hydrocarbon radical having the specified number of carbon atoms. Thus, “(C₁-C₆)alkyl” means a radical having from 1-6 carbon atoms in a linear or branched arrangement. “(C₁-C₆)alkyl” includes, but is not limited to: methyl, ethyl, n-propyl, i-propyl, n-butyl, i-butyl, t-butyl, n-pentyl, i-pentyl, and n-hexyl.

“Cycloalkyl” means a saturated aliphatic cyclic hydrocarbon radical having the specified number of carbon atoms. Thus, (C₃-C₆)cycloalkyl means a radical having from 3-6 carbon atoms arranged in a ring. (C₃-C₆)cycloalkyl includes cyclopropyl, cyclobutyl, cyclopentyl, and cyclohexyl.

Haloalkyl includes mono, poly, and perhaloalkyl groups where the halogens are independently selected from fluorine, chlorine, and bromine.

“Heteroaryl” means a monovalent heteroaromatic monocyclic and polycyclic ring radical. Heteroaryl rings are 5- and 6-membered aromatic heterocyclic rings containing 1 to 4 heteroatoms independently selected from N, O, and S, and include furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole. Bicyclic heteroaryl rings are bicyclo[4.4.0] and bicyclo[4,3.0] fused ring systems containing 1 to 4 heteroatoms independently selected from N, O, and S, and include indolizine, indole, isoindole, benzo[b]furan, benzo[b]thiophene, indazole, benzimidazole, benzthiazole, purine, 4H-quinolizine, quinoline, isoquinoline, cinnoline, phthalzine, quinazoline, quinoxaline, 1,8-naphthyridine, and pteridine.

“Alkoxy” means an alkyl radical attached through an oxygen linking atom. “(C₁-C₄)alkoxy” includes methoxy, ethoxy, n-propoxy, i-propoxy, n-butoxy, i-butoxy, and t-butoxy.

“Cycloalkoxy” means a cycloalkyl radical attached through an oxygen linking atom. “(C₃-C₆)cycloalkoxy” includes cyclopropyloxy, cyclobutyloxy, cyclopentyloxy, and cyclohexyloxy.

The present invention is directed to the ruthenium catalyzed hydrogenation of ketones of Formula (II) to selectively provide alcohols of Formula (I) as depicted in the following Scheme:

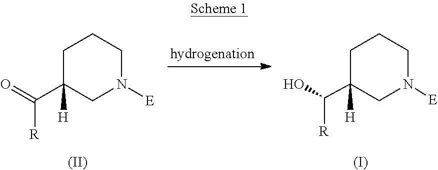

In a preferred embodiment of the invention, the ruthenium catalyst is (S)-2-[(Sₚ)-2-(diphenylphosphino)ferrocenyl]-4-isopropyl-2-oxazoline triphenylphosphine ruthenium(II) chloride complex.

In another preferred embodiment of the invention, the base is aqueous sodium hydroxide in a concentration range of from about 0.01M to about 1M, more preferably about 1M. In a further preferred embodiment of the invention, the mole ratio of sodium hydroxide to ruthenium catalyst is in the range of from about 15:1 to about 5:1; more preferably, the mole ratio of sodium hydroxide to ruthenium catalyst is about 15:1 (e.g. 13:1-17:1).

The hydrogenation reaction is conducted in an appropriate solvent system that is inert to the reaction conditions. The term solvent system is used to indicate that a single solvent or alternatively a mixture of two or more solvents can be used. The term inert is used to mean that the solvent system does not react unfavorably with the reactants, products, or the catalyst. The solvent system need not completely dissolve the ketone reactant or the chiral alcohol product. In a preferred embodiment of the invention, the hydrogenation reaction is conducted in a solvent selected from the group consisting of 2-butanol, methyl tert-butyl ether, cyclopentyl methyl ether, toluene, 2-methyltetrahydrofuran, dichloromethane, and mixtures thereof; more preferably, the hydrogenation reaction is conducted in methyl tert-butyl ether or toluene.

The term hydrogenation, as used herein, refers to reaction of the ketone with a source of hydrogen atoms under appropriate conditions so that two hydrogen atoms are added to the carbonyl group of the ketone to produce the hydroxyl group of the chiral alcohol. Preferably the source of hydrogen atoms includes molecular hydrogen (H₂ gas). In a preferred embodiment of the invention, the hydrogen gas pressure is in the range of from about 4 to about 18 bar, more preferably the hydrogen gas pressure is about 4 bar (e.g. 4 bar±0.5 bar).

The temperature during the reaction may in principle be chosen arbitrarily by the person skilled in the art, as long as a sufficiently quick and selective reaction is achieved. However, it should be taken into account that the choice of solvent and stability of the catalyst will have an effect on the temperature chosen. In a preferred embodiment of the invention, the reaction is conducted at a temperature of about 25° C. (e.g. 21-27° C.).

In a specific embodiment of the invention, R is 3-chlorophenyl and E is tert-butoxycarbonyl.

A further specific embodiment of the invention is directed to a process for the asymmetric preparation of tert-butyl (3R)-3-[(R)-(3-chlorophenyl)(hydroxy)-methyl]-1-piperidinecarboxylate represented by Formula (Ia):

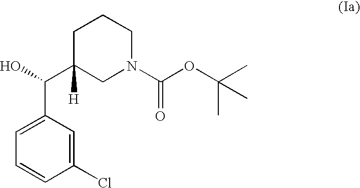

comprising hydrogenating a ketone of Formula (Ia):

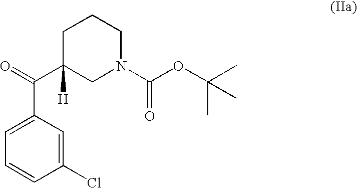

under 4 bar hydrogen gas pressure, in methyl tert-butyl ether at 25° C., in the presence of 1M aqueous sodium hydroxide and (S)-2-[(Sₚ)-2-(diphenylphosphino)-ferrocenyl]-4-isopropyl-2-oxazoline triphenylphosphine ruthenium(II) chloride complex.

The process of this invention provides alcohol (Ia) in high yield and high enantiomeric and diastereomeric purity (high e.e. and d.e.).

The invention is further defined by reference to the examples, which are intended to be illustrative and not limiting.

Representative compounds of the invention can be synthesized in accordance with the general reaction conditions described above and are illustrated in the examples that follow. The methods for preparing the various starting materials used in the examples are well within the knowledge of persons skilled in the art. Ketone substrates of formula (II) are known and can be prepared as described in International Publications WO 2006/042150, WO 2007/070201, WO 2007/117557, WO 2007/117560, WO 2008/036216, and WO 2008/036247, or by any other suitable method readily apparent to the person skilled in the art.

### EXAMPLE 1

### tert-butyl (3R)-3-[(R)-(3-chlorophenyl)(hydroxy)-methyl]-1-piperidinecarboxylate

tert-Butyl (3R)-3-[(3-chlorophenyl)carbonyl]-1-piperidinecarboxylate (5.70 g, 17.6 mmol) (prepared by the method described in WO 2008/036247) and (S)-2-[(Sₚ)-2-(diphenylphosphino)ferrocenyl]-4-isopropyl-2-oxazoline triphenylphosphine ruthenium(II) chloride complex (0.322 g, 0.352 mmol) were combined in a Parr reactor vessel. Methyl tert-butyl ether (57 mL) and 1 M aqueous sodium hydroxide (5.7 mL) were added sequentially. The vessel was purged three times with H₂ and then pressurized with 4 bar H₂. The reaction mixture was stirred at 250 rpm for 22 h at ambient temperature at which point Ion Pair Chromatography (LC@220 nm) showed 99.8% conversion. The reaction mixture was washed with water (50 mL) and the crude organic layer was passed through a SiO₂ plug to remove the catalyst. The methyl tert-butyl ether was removed under reduced pressure to give a solid (5.5 g crude, 85.8% d.e. and 98% e.e.). The crude solid was recrystallized by dissolving in methyl tert-butyl ether (30 mL) and heptane (50 mL) with stirring, followed by the addition of seed crystals (10 mg, prepared by a method as generally described in WO 2008/036247). Stirring was continued for 15 min. as a white precipitate formed. The solution was cooled to 0° C. and further treated with heptane (30 mL). The crystals were collected by filtration, washed with cold heptane (3×50 mL), and dried under vacuum to give the title compound (3.4 g, 60%) as a white solid (100% d.e., 99.7% e.e.). ¹H NMR (400 MHz, d₃-MeCN) δ 7.35-7.24 (m, 4H), 4.35 (dd, J=7.2 Hz, 4.4 Hz, 1H), 4.05 (d, J=11.7 Hz, 1H), 3.85 (d, 12.9 Hz, 1H), 3.45 (s, 1H), 2.68-2.65 (m, 2H), 2.14 (s, 1H), 1.65-1.56 (m, 2H), 1.38 (s, 9H), 1.34-1.14 (m, 2H); ¹³C NMR (125 MHz, d₃-MeCN) δ 155.7, 147.6, 134.6, 130.8, 128.2, 127.5, 126.1, 100.8, 79.7, 75.9, 44.9, 44.2, 28.6, 28.3, 25.7; IR (solid, cm⁻¹) 3450, 1664, 1418, 1142, 792; LRMS (API-ES, Pos. Ion) m/z 673 [2M+Na]⁺, 389 [M+ACN+Na]⁺, 348 [M+Na]⁺, 293, 252. Enantio- and diastereoselectivity were determined by HPLC using a Chiracel AD-H column eluting with 95% hexanes/5% ethanol (isocratic) at 1 mL/min. with detection@215 nm. Diastereoselectivity and conversion were also determined by achiral HPLC using a Zorbax Eclipse SB column.

What is claimed is:

1. A process for the preparation of a compound of Formula (I): 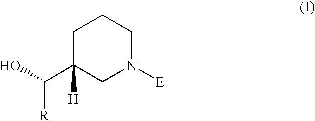
wherein R is phenyl optionally substituted with 1 to 3 groups independently selected from:
1) halogen, (C₁-C₆)alkyl, (C₃-C₆)cycloalkyl, halo(C₁-C₆)alkyl, (C₁-C₆)alkoxy, and (C₃-C₆)cycloalkoxy; and
2) phenyl, heteroaryl, phenoxy, and heteroaryloxy, each optionally substituted with 1 to 2 groups independently selected from: halogen, (C₁-C₄)alkyl, halo(C₁-C₄)alkyl, (C₁-C₄)-alkoxy, and (C₁-C₄)alkoxy(C₁-C₄)alkyl; and
E is an amine protecting group;
said process comprising hydrogenating a ketone of Formula (II): 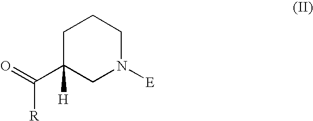
under hydrogen gas pressure in the presence of a base and a ruthenium catalyst.

2. The process according to claim 1, wherein the ruthenium catalyst is (S)-2-[(Sₚ)-2-(diphenylphosphino)ferrocenyl]-4-isopropyl-2-oxazoline triphenylphosphine ruthenium(II) chloride complex.

3. The process according to claim 1, wherein the base is 1M aqueous sodium hydroxide.

4. The process according to claim 3, wherein the mole ratio of sodium hydroxide to ruthenium catalyst is in the range of from 15:1 to 5:1.

5. The process according to claim 4, wherein the mole ratio of sodium hydroxide to ruthenium catalyst is 15:1.

6. The process according to claim 1, wherein the hydrogenation reaction is conducted in a solvent which is methyl tert-butyl ether or toluene.

7. The process according to claim 1, wherein the hydrogen gas pressure is in the range of from 4 to 18 bar.

8. The process according to claim 7, wherein the hydrogen gas pressure is 4 bar.

9. The process according to claim 1, wherein the hydrogenation reaction is conducted at a temperature in the range of from 21 to 27° C.

10. The process according to claim 1, wherein R is 3-chlorophenyl and E is tert-butoxycarbonyl.

11. A process for the preparation of tert-butyl (3R)-3-[(R)-(3-chlorophenyl)(hydroxy)-methyl]-1-piperidinecarboxylate represented by Formula (Ia): 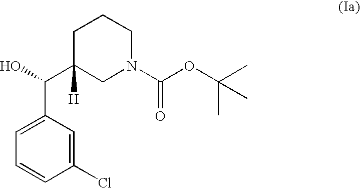
comprising hydrogenating a ketone of Formula (IIa): 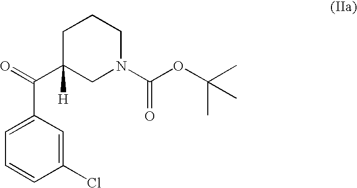
under 4 bar hydrogen gas pressure, in methyl tert-butyl ether at 25° C., in the presence of 1M aqueous sodium hydroxide and (S)-2-[(Sₚ)-2-(diphenylphosphino)ferrocenyl]-4-isopropyl-2-oxazoline triphenylphosphine ruthenium(II) chloride complex.
